(19) United States
(12) Patent Application Publication
(10) Pub. No.: US 2022/0193176 A1
(43) Pub. Date: Jun. 23, 2022

(54) COMPOSITION OF CHINESE MEDICAL HERB FOR INHIBITING COVID-19 VIRUS, USE OF SAME, AND METHOD OF MAKING SAME

(71) Applicant: XIU WUCHEN BIOTECHNOLOGY CO., LTD., Taipei City (TW)

(72) Inventors: PEI-FEN WU, Taipei City (TW); MING-CHI SHIH, Taipei City (TW)

(21) Appl. No.: 17/151,696

(22) Filed: Jan. 19, 2021

(30) Foreign Application Priority Data
Dec. 18, 2020 (TW) 109145112

(51) Int. Cl. A61K 36/899; A61K 36/537; A61K 36/355; A61K 36/78; A61K 36/752; A61P 31/14

(52) U.S. Cl. CPC A61K 36/899; A61K 36/537; A61K 36/355; A61K 2236/51; A61K 36/752; A61P 31/14; A61K 2236/333; A61K 36/78

(57) ABSTRACT

A composition of Chinese medical herbs capable of inhibiting COVID-19 virus, use of the same, and method of making the same are provided. The composition of Chinese medical herbs includes Pennisetum alopecuroides, Lophatherum gracile, Salvia miltiorrhiza, Lonicera japonica, Houttuynia cordata and Tangerine Peel and is suitable for making a drug capable of inhibiting COVID-19. The method includes: providing Pennisetum alopecuroides, Lophatherum gracile, Salvia miltiorrhiza, Lonicera japonica, Houttuynia cordata and Tangerine Peel; extracting the Chinese medical herbs with an organic solvent to produce an extract; and removing the organic solvent from the extract to obtain the composition of Chinese medical herbs. The composition of Chinese medical herbs suppresses the activity of COVID-19 virus.

### CROSS-REFERENCE TO RELATED APPLICATION

This non-provisional application claims priority under 35 U.S.C. § 119(a) on Patent Application No(s). 109145112 filed in Taiwan, R.O.C. on Dec. 18, 2020, the entire contents of which are hereby incorporated by reference.

### BACKGROUND OF THE INVENTION

### 1. Field of the Invention

The present disclosure relates to a composition of Chinese medical herbs, use of the same, and method of making the same, and in particular to a composition of Chinese medical herbs capable of inhibiting COVID-19 virus.

### 2. Description of the Related Art

COVID-19 (Coronavirus Disease 2019) spreads quickly worldwide and is caused by Severe Acute Respiratory Syndrome Coronavirus 2 (SARS-CoV-2). SARS-CoV-2 is a new analog of SARS-CoV and MERS virus and is transmitted via oral and nasal secretions.

### BRIEF SUMMARY OF THE INVENTION

So far no drug has been developed to effectively treat COVID-19. Although small molecule drugs which are used in treating other diseases served as makeshift medication for COVID-19, these drugs demonstrate no proven efficacy in treating COVID-19.

To achieve at least the above objective, the present disclosure provides a composition of Chinese medical herbs for inhibiting COVID-19 virus, comprising Pennisetum alopecuroides, Lophatherum gracile, Salvia miltiorrhiza, Lonicera japonica, Houttuynia cordata and Tangerine Peel.

The composition of Chinese medical herbs comprises 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel.

To achieve at least the above objective, the present disclosure provides a use of a composition of Chinese medical herbs for use in making a drug for inhibiting COVID-19 virus, comprising the composition of Chinese medical herbs.

To achieve at least the above objective, the present disclosure provides a method of making a composition of Chinese medical herbs for inhibiting COVID-19 virus, comprising the steps of: (a) providing 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel; (b) extracting the 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel provided in step (a) with an organic solvent to produce an extract; and (c) removing the organic solvent from the extract to obtain the composition of Chinese medical herbs.

Regarding the method, the organic solvent used in step (b) is 70% volume percent of ethanol, and weight ratio of the ethanol to the 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel provided in step (a) is 20:1.

Regarding the method, step (c) entails performing a centrifugal process on the extract at rotational speed of 4000-5000 rpm to obtain a supernatant, concentrating the supernatant, and drying the supernatant to remove the organic solvent from the supernatant.

Regarding the method, the concentrating and drying processes of step (c) entails freezing and drying the supernatant.

The composition of Chinese medical herbs, use of the same, and method of making the same are effective in inhibiting SARS-CoV-2 which causes COVID-19.

### BRIEF DESCRIPTION OF THE DRAWINGS

FIG. 1 shows results of pseudovirus neutralization tests performed on composition EC-51 of Chinese medical herbs according to an embodiment of the present disclosure.

FIG. 2 illustrates the inhibitory effect of the composition EC-51 of Chinese medical herbs on the spike protein of SARS-CoV-2 bound to ACE2 receptors according to an embodiment of the present disclosure.

FIG. 3 illustrates the cellular toxicity test result of the composition EC-51 of Chinese medical herbs according to an embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

Making Composition EC-51 of Chinese Medical Herbs

First, provide 300 g of Pennisetum alopecuroides, 100 g of Lophatherum gracile, 200 g of Salvia miltiorrhiza, 75 g of Lonicera japonica, 125 g of Houttuynia cordata and 200 g of Tangerine Peel (i.e., 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel) which together weigh 1 kg. Then, use 20 kg of 70% volume percent of ethanol as solvent (i.e., weight ratio of the ethanol to the Chinese medical herbs is 20:1). Next, dissolving the Chinese medical herbs in the 70% ethanol, obtaining an extract from the Chinese medical herbs at 60° C., performing a centrifugal process on the extract at rotational speed of 4500 rpm for 15 minutes to obtain a supernatant which comprises the composition of Chinese medical herbs, concentrating and drying the supernatant at low temperature and vacuum to remove the organic solvent from the supernatant, so as to obtain the composition of Chinese medical herbs. After that, around 150 g of powder formulation is made from the composition of Chinese medical herbs (the yield is around 13% to 15% in this embodiment). The powder formulation is made from the composition of Chinese medical herbs by freezing and drying the supernatant. The inventor of the present disclosure names the resultant composition of Chinese medical herbs EC-51.

In another embodiment, the respective weights of the Chinese medical herbs may be subject to changes as needed, provided that the respective parts by weight of the Chinese medical herbs remain unchanged.

In this embodiment, the composition of Chinese medical herbs undergoes extraction by distillation. However, in another embodiment, the composition of Chinese medical herbs undergoes extraction by column chromatography or with supercritical fluid (SCF) as needed.

In this embodiment, the 70% ethanol serves as the solvent. In another embodiment, the solvent is a conventional organic solvent for use in extraction of Chinese medical herbs, such as methanol and acetone, wherein the distillation temperature and weight ratio of the organic solvent to Chinese medical herbs are adjustable according to the chemical and physical properties of the organic solvent.

In this embodiment, the extract undergoes a centrifugal process at rotational speed of 4500 rpm to obtain the composition of Chinese medical herbs. However, in another embodiment, the centrifugal process may take place at rotational speed of 4000-5000 rpm or at any appropriate rotational speed, provided that the composition of Chinese medical herbs is eventually separated from the extract. In another embodiment, the centrifugal process is performed with any conventional means, other than a means for centrifugal separation, for extracting the composition of Chinese medical herbs from the extract and removing an organic solvent therefrom, such as column chromatography, as needed.

Neutralization Test Performed on SARS-CoV-2 Pseudovirus with Composition EC-51 of Chinese Medical Herbs

Both HeLa cells (commercially available) which express SmBiT-ACE2 and SARS-CoV-2 pseudovirus (commercially available) are first prepared. Then, eight samples of HeLa cells which express SmBiT-ACE2 and eight SARS-CoV-2 pseudovirus samples are treated with eight EC-51 formulas with concentrations of 1000 μg/ml, 500 μg/ml, 250 μg/ml, 125 μg/ml, 63 μg/ml, 31 μg/ml, 16 μg/ml and 8 μg/ml (dissolved in dimethyl sulfoxide (DMSO)) at 37° C. for three hours to obtain eight experiment group samples (each experiment group sample contains an EC-51-treated group of HeLa cells which express SmBiT-ACE2 and a group of SARS-CoV-2 pseudovirus). The group of HeLa cells which express SmBiT-ACE2 and a group of SARS-CoV-2 pseudovirus are treated with DMSO which has a volume equivalent to the above EC-51 formulas at 37° C. for three hours to obtain control group sample.

Then, a 96-well cell culture plate is provided, and the eight experiment group samples (which contain 0.1 ml of MEM liquid culture medium, wherein both the HeLa cells which express SmBiT-ACE2 and the SARS-CoV-2 pseudovirus, which are treated with EC-51 with different concentrations, are contained in the MEM liquid culture medium, wherein the HeLa cells are of concentration of 1×10⁵ cells/ml, and the viral concentration is 0.01 MOI) and control group sample (which contain 0.1 ml of MEM liquid culture medium, wherein both the HeLa cells which express SmBiT-ACE2 and the SARS-CoV-2 pseudovirus, which are treated with DMSO, are contained in MEM liquid culture medium, wherein the HeLa cells are of concentration of 1×10⁵ cells/ml, and viral concentration is 0.01 MOI) are introduced into nine of the wells of the 96-well cell culture plate. After that, the cell culture plate which holds the eight experiment group samples and control group sample undergoes culturing at 37° C. for 48 hours, such that the SARS-CoV-2 pseudovirus infects the HeLa cells.

After the eight experiment group samples and control group sample have undergone culturing at 37° C. for 48 hours, the cells in the eight experiment group samples and control group sample are rinsed with phosphate buffered saline (PBS) solution. Finally, Nanoluc reagent is used to assay the level of fluorescence expressed by the HeLa cells which express SmBiT-ACE2, so as to calculate the inhibition rate of the composition EC-51 of Chinese medical herbs on the SARS-CoV-2 pseudovirus. The test is carried out thrice to obtain the average experiment result.

Principle of the Test

After SARS-CoV-2 pseudovirus bound to ACE2 receptors of HeLa cells has entered the HeLa cells, the HeLa cells express luciferase, because SARS-CoV-2 pseudovirus carries a luciferase reporter gene. The luciferase reacts with fluorogenic substrate of Nanoluc reagent to produce fluorescence. Thus, by detecting the fluorescence level of samples, it is feasible to evaluate the extent to which the SARS-CoV-2 pseudovirus infects the HeLa cells. Neutralization can occur between the EC-51 and the SARS-CoV-2 pseudovirus; thus, the EC-51 can prevent the SARS-CoV-2 pseudovirus from infecting the HeLa cells. With the SARS-CoV-2 pseudovirus being denied to entry into the HeLa cells, there is no production of luciferase which will otherwise react with fluorogenic substrate in Nanoluc reagent. As a result, the level of the fluorescence expressed in the samples decreases, indicating that the SARS-CoV-2 pseudovirus infects the HeLa cells to a lesser extent. Therefore, the inhibition rate of the composition EC-51 of Chinese medical herbs on the SARS-CoV-2 pseudovirus is calculated according to the level of the fluorescence expressed by the HeLa cells.

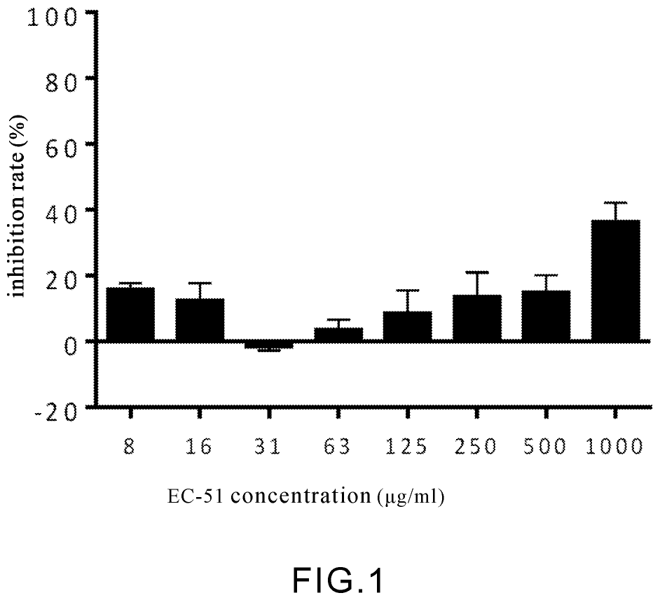

As indicated by the experiment result shown in Table 1 and FIG. 1, neutralization between the composition EC-51 of Chinese medical herbs and the SARS-CoV-2 pseudovirus is substantially dose-dependent, that is, the higher the concentration of EC-51, the better its inhibitory effect on SARS-CoV-2 pseudovirus, especially when the concentration of the composition EC-51 of Chinese medical herbs ranges from 31 μg/ml to 1000 μg/ml. The inhibition rate of the composition EC-51 of Chinese medical herbs on the SARS -CoV-2 pseudovirus is 37% when the concentration of the composition EC-51 of Chinese medical herbs is 1000 μg/ml. Since the capsid of the SARS-CoV-2 pseudovirus has the same spike protein as SARS-CoV-2 does, the experiment result infers satisfactory neutralization between the composition EC-51 of Chinese medical herbs and the SARS-CoV-2, thereby stopping the SARS-CoV-2 from entering the cells.

TABLE 1
result of inhibition rate of neutralization test on composition
EC-51 of Chinese medical herbs on SARS-CoV-2 pseudovirus

| | pseudovirus inhibition rate (%) | | | |
|---|---|---|---|---|
| EC-51 | | | | average |
| concentration | experiment | experiment | experiment | inhibition |
| μg/ml | 1 | 2 | 3 | rate |
| 1000 | 32.3 | 29.8 | 47.7 | 36.6 |
| 500 | 4.9 | 21.9 | 18.2 | 15.0 |
| 250 | 19.3 | 22.5 | −0.3 | 13.8 |
| 125 | 16.5 | 14.3 | −4.3 | 8.9 |
| 63 | 3.6 | 8.9 | −1.1 | 3.8 |
| 31 | −1.8 | 0.4 | −3.4 | −1.6 |
| 16 | 4.9 | 22.1 | 11.0 | 12.7 |
| 8 | 15.2 | 19.2 | 13.7 | 16.0 |

Inhibitory effect of the composition EC-51 of Chinese medical herbs on the SARS-CoV-2 whose spike protein is bound to ACE2 receptors is described below.

A protein-ligand complex composed of a receptor-binding domain (RBD) of the spike protein of the SARS-CoV-2 and an LgBiT fusion protein (abbreviated as RBD-LgBiT, which is commercially available) is first prepared. Then, eight RBD-LgBiT are co-incubated with eight EC-51 formulas (dissolved in dimethyl sulfoxide, DMSO) with concentrations of 1000 μg/ml, 500 μg/ml, 250 μg/ml, 125 μg/ml, 63 μg/ml, 31 μg/ml, 16 μg/ml and 8 μg/ml and eight RBD-LgBiT at room temperature for 15 minutes to form eight RBD-LgBiT which have been treated with the EC-51 formulas of different concentrations to serve as eight experiment group samples. Furthermore, DMSO of the same volume as the EC-51 formulas and another group of RBD-LgBiT are co-incubated at room temperature for 15 minutes to serve as a control group sample.

Then, a 96-well cell culture plate is provided. Next, 0.1 ml of MEM liquid culture medium is introduced into nine of the wells each of the 96-well cell culture plate. The liquid culture medium contains HeLa cells (1×10⁵ cells/ml) which express SmBiT-ACE2 (commercially available). After that, the eight experiment group samples (which contain the RBD-LgBiT treated with the EC-51 of different concentrations) and control group sample (which contains the DMSO-treated RBD-LgBiT) are introduced into the nine groups of HeLa cells. Furthermore, NanoGlo reagent substrate is introduced into the nine groups of HeLa cells to facilitate a subsequent test. The amount of the NanoGlo reagent substrate introduced into the nine groups of HeLa cells is based on the description of the user manual for Nano-glo live cell assay reagent kit.

Then, let the cell culture plate stand still at 37° C. for 24 hours, such that RBD-LgBiT binds to the HeLa cells which express SmBiT-ACE2. After the cell culture plate has stood still for 24 hours, the binding reaction between the RBD-LgBiT and the HeLa cells is assayed. The rationale is: when the LgBiT protein of the RBD-LgBiT binds to the SmBiT protein on the HeLa cells which express SmBiT-ACE2, it means that the RBD-LgBiT enters into the cells through the ACE2 receptors on the HeLa cells which express SmBiT-ACE2 via the RBD protein; meanwhile, the LgBiT and the SmBiT together form luciferase by their structural complementary fusion, and the LgBiT-SmBiT luciferase reacts with the NanoGlo reagent substrate to generate fluorescence. Finally, the level of fluorescence expressed by the eight experiment group samples and control group sample is assayed with a microplate reader to thereby evaluate the binding of the RBD-LgBiT to the HeLa cells expressing SmBiT-ACE2 in each sample, so as to calculate the inhibition rate of the composition EC-51 of Chinese medical herbs on the SARS-CoV-2 whose spike protein is bound to ACE2 receptors. Cell survival rate of the HeLa cells in the eight experiment group samples is also calculated. This test is carried out thrice to obtain the average experiment result.

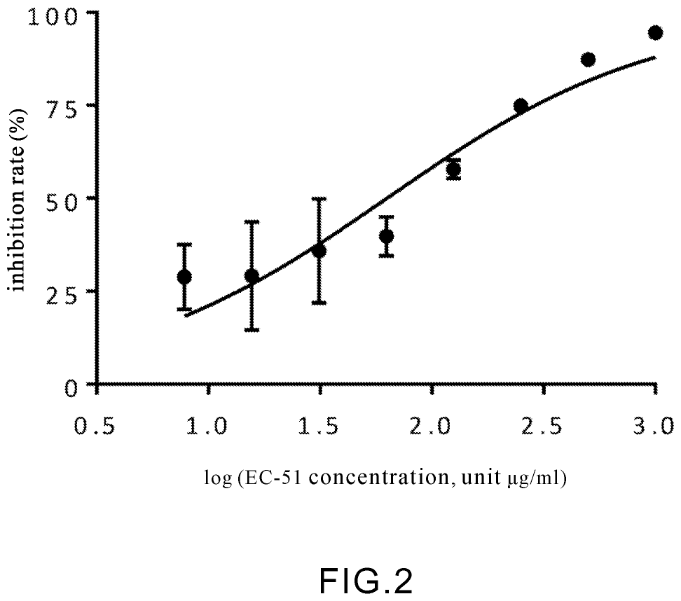

As indicated by the experiment result shown in Table 2 and FIG. 2, the inhibition rate of the composition EC-51 of Chinese medical herbs on the SARS-CoV-2 whose spike protein is bound to ACE2 receptors and the cell survival rate of the HeLa cells bound with spike protein of SARS-CoV-2 and treated with the composition EC-51 of Chinese medical herbs have a dose-dependent relationship, that is, the higher the concentration of EC-51 is, the better is its inhibitory effect on the SARS-CoV-2 whose spike protein is bound to ACE2 receptors, and the higher is the cell survival rate. The concentration (i.e., EC50) of the composition EC-51 of Chinese medical herbs required to inhibit 50% of the receptor-binding domain bound to the HeLa cells is calculated to be 62.9 μg/ml.

TABLE 2
survival rate of cells bound to spike protein of
SARS-CoV-2 and treated with composition EC-51 of
Chinese medical herbs of different concentrations

| EC-51 | cell survival rate (%) | | | |
|---|---|---|---|---|
| concentration | experiment | experiment | experiment | average |
| μg/ml | 1 | 2 | 3 | survival rate |
| 1000 | 94.7 | 94.6 | 94.4 | 94.6 |
| 500 | 87.4 | 86.9 | 87.5 | 87.3 |
| 250 | 76.3 | 74.4 | 73.9 | 74.9 |
| 125 | 57.3 | 55.7 | 60.5 | 57.8 |
| 63 | 36.5 | 37.0 | 45.8 | 39.8 |
| 31 | 27.4 | 28.2 | 52.0 | 35.8 |
| 16 | 22.4 | 19.1 | 45.8 | 29.1 |
| 8 | 27.3 | 21.0 | 38.2 | 28.8 |

The receptor-binding domain of the spike protein of the SARS-CoV-2 binds to the ACE2 receptors on a cell in order for the SARS-CoV-2 to enter the cell. As indicated by the experiment result, the neutralization between EC-51 and the receptor-binding domain of the spike protein of the SARS-CoV-2 prevents the SARS-CoV-2 from infecting the human body.

Toxicity Test on Composition EC-51 of Chinese Medical Herbs with Respect to HeLa Cells

First, a 96-well cell culture plate is provided, and 0.1 ml of MEM liquid culture medium is introduced into 14 wells of the 96-well cell culture plate as fourteen groups of HeLa cells. The liquid culture medium contains HeLa cells. (the HeLa cells are of concentration of 1×10⁵ cells/ml and is commercially available.) Eight groups of HeLa cells serve as eight experiment group samples for this test. Six groups of HeLa cells serve as six control group samples for this test. Then, EC-51 formulas (dissolved in dimethyl sulfoxide, DMSO) are introduced into the eight experiment group samples, such that the concentrations of the EC-51 formulas in the eight experiment group samples are 2000 μg/ml, 1111 μg/ml, 617 μg/ml, 342 μg/ml, 190 μg/ml, 105 μg/ml, 59 μg/ml and 33 μg/ml. Furthermore, DMSO is introduced into the six control group samples, such that the concentrations of the DMSO formulas in the six control group samples are 2000 μg/ml, 1111 μg/ml, 617 μg/ml, 342 μg/ml, 190 μg/ml and 59 μg/ml. After that, the experiment group samples and control group samples are kept at 37° C. for 72 hours. Next, the cell survival rate of the HeLa cells in the experiment group samples and control group samples is assayed with CellTiter-Glo Luminescent reagent, so as to evaluate the toxicity tolerance of the HeLa cells toward the composition EC-51 of Chinese medical herbs. This test is carried out thrice to obtain the average experiment result.

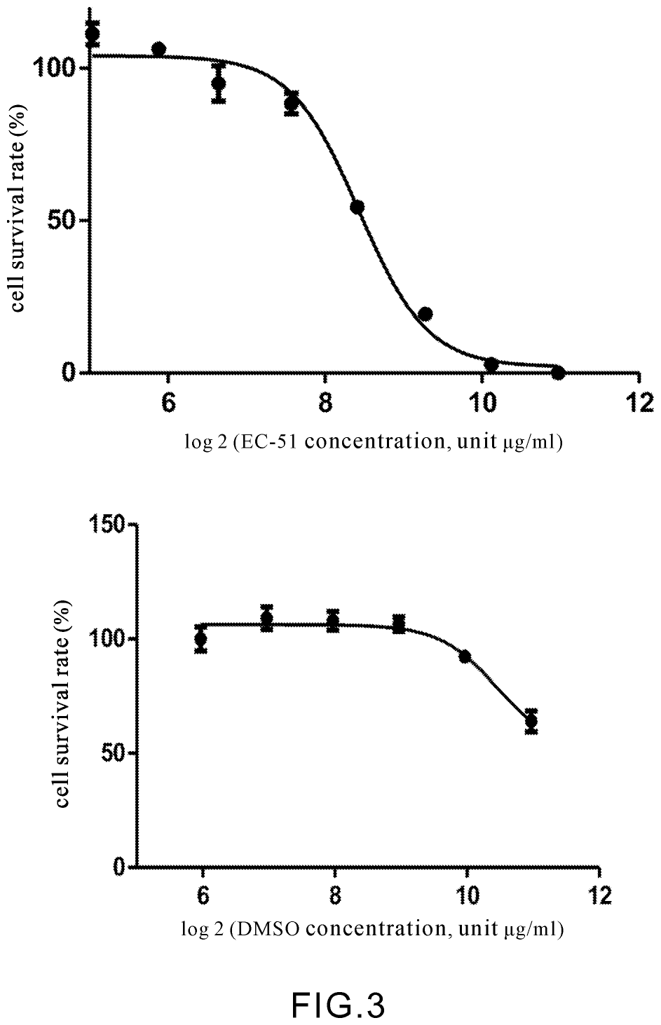

As indicated by the experiment result shown in Table 3 and FIG. 3, the safety dosage of EC-51 is calculated to be 160 μg/ml or less in accordance with the experiment result, such that a 90% cell survival rate can be achieved by administering EC-51 of a concentration 160 μg/ml or less to the cells. Thus, IC₅₀ of cellular toxicity of the composition EC-51 of Chinese medical herbs is calculated to be 346 μg/ml. The composition EC-51 of Chinese medical herbs is applicable to the human body and thus suitable for making a drug for inhibiting COVID-19 virus.

TABLE 3
cell survival rate of HeLa cells treated with EC-51 of different
concentrations to undergo cellular toxicity test

| EC-51 | cell survival rate (%) | | | |
|---|---|---|---|---|
| concentration | experiment | experiment | experiment | average |
| μg/ml | 1 | 2 | 3 | survival rate |
| 2000 | 0 | 0 | 0 | 0 |
| 1111 | 3 | 3 | 3 | 3 |
| 617 | 19 | 20 | 18 | 19 |
| 342 | 51 | 54 | 58 | 54 |
| 190 | 89 | 82 | 94 | 88 |
| 105 | 90 | 89 | 107 | 95 |
| 59 | 108 | 103 | 108 | 106 |
| 33 | 109 | 106 | 118 | 111 |

Therefore, the composition EC-51 of Chinese medical herbs not only undergoes satisfactory neutralization with respect to SARS -CoV-2 but also inhibits SARS-CoV-2 whose spike protein is bound to ACE2 receptors, such that the composition EC-51 of Chinese medical herbs can stop the SARS-CoV-2 from entering the cells. In addition, the composition EC-51 of Chinese medical herbs is applicable to the human body and thus suitable for making a drug for inhibiting COVID-19 virus.

While the present disclosure has been described by means of specific embodiments, numerous modifications and variations could be made thereto by those skilled in the art without departing from the scope and spirit of the present disclosure set forth in the claims.

What is claimed is:

1. A composition of Chinese medical herbs for inhibiting COVID-19 virus, comprising Pennisetum alopecuroides, Lophatherum gracile, Salvia miltiorrhiza, Lonicera japonica, Houttuynia cordata and Tangerine Peel.

2. The composition of Chinese medical herbs of claim 1, comprising 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel.

3. A use of a composition of Chinese medical herbs for use in making a drug for inhibiting COVID-19 virus, comprising the composition of Chinese medical herbs of claim 1.

4. A use of a composition of Chinese medical herbs for use in making a drug for inhibiting COVID-19 virus, comprising the composition of Chinese medical herbs of claim 2.

5. A method of making a composition of Chinese medical herbs for inhibiting COVID-19 virus, comprising the steps of:
(a) providing 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel;
(b) extracting the 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel provided in step (a) with an organic solvent to produce an extract; and
(c) removing the organic solvent from the extract to obtain the composition of Chinese medical herbs.

6. The method of claim 5, wherein the organic solvent used in step (b) is 70% volume percent of ethanol, and weight ratio of the ethanol to the 12 parts by weight of Pennisetum alopecuroides, 4 parts by weight of Lophatherum gracile, 8 parts by weight of Salvia miltiorrhiza, 3 parts by weight of Lonicera japonica, 5 parts by weight of Houttuynia cordata and 8 parts by weight of Tangerine Peel provided in step (a) is 20:1.

7. The method of claim 5, wherein step (c) entails performing a centrifugal process on the extract at rotational speed of 4000-5000 rpm to obtain a supernatant, concentrating the supernatant, and drying the supernatant to remove the organic solvent from the supernatant.

8. The method of claim 7, wherein the concentrating and drying processes of step (c) entails freezing and drying the supernatant.
